(19) United States
(12) Patent Application Publication
(10) Pub. No.: US 2025/0281108 A1
(43) Pub. Date: Sep. 11, 2025

(54) PRESSURE ULCER PREVENTION BED USING BODY PRESSURE SENSORS AND CONTROL METHOD THEREOF

(71) Applicant: Ninebell Healthcare Co., Ltd., Seongnam-si (KR)

(72) Inventors: Young Dae LEE, Seoul (KR); Yun Hee JEONG, Yeoju-si (KR)

(73) Assignee: Ninebell Healthcare Co., Ltd., Seongnam-si (KR)

(21) Appl. No.: 18/764,366

(22) Filed: Jul. 5, 2024

(30) Foreign Application Priority Data
Mar. 11, 2024 (KR) 10-2024-0033897

(51) Int. Cl. A61B 5/00; A61G 7/015; A61G 7/018; A61G 7/057

(52) U.S. Cl. CPC A61B 5/447; A61G 7/015; A61G 7/018; A61G 7/0573; A61B 2562/0247; A61G 2203/70

(57) ABSTRACT

A pressure ulcer prevention bed using body pressure sensors and a control method thereof are proposed. The pressure ulcer prevention bed includes a plurality of raising and lowering link modules arranged in parallel adjacent to an upper surface of a bed support unit and whose raising and lowering is independently controlled by parallelogram links, the body pressure sensors provided in the respective raising and lowering link modules and constantly collecting body pressure sensing information for each part of a human body applied to each raising and lowering link module, a servo motor provided in the parallelogram links of each raising and lowering link module, and a motion control unit connected to the servo motor through wired and wireless communication and independently controlling each raising and lowering link module by using the body pressure sensing information individually transmitted from the body pressure sensors.

### CROSS REFERENCE TO RELATED APPLICATION

The present application claims priority to Korean Patent Application No. 10-2024-0033897, filed Mar. 11, 2024, the entire contents of which are incorporated by reference.

### BACKGROUND

### Field of the Disclosure

The present disclosure relates to a pressure ulcer prevention bed and a control method thereof using body pressure sensors and, more particularly, to a pressure ulcer prevention bed and a control method thereof using body pressure sensors, wherein a plurality of raising and lowering link modules (i.e., keyboards) in a multi-parallelogram link structure in which raising and lowering is independently controlled in the form of raising and lowering link modules is arranged in parallel adjacent to each other along a surface of a bed, a plurality of body pressure sensors is arranged at predetermined intervals on the keyboards, and then the raising and lowering movements of the keyboards are appropriately controlled according to measured values of the body pressure sensors, so that it is highly effective in preventing pressure ulcers, skin diseases, etc.

### Background

In general, medical beds used in hospitals are structured such that a back support or foot support thereof is rotatable and foldable at a predetermined angle for patients with limited mobility, and are classified into the manual medical beds in which a guardian adjusts the angle of the back support by turning an operation handle and the electric medical beds in which the guardian adjusts the angle of the back support with electric power by using a drive motor or hydraulic cylinder.

The manual medical beds have an advantage in that it is inexpensive in terms of not requiring a separate motorized device, but have a problem in that it is difficult for the patients with limited mobility to use the manual medical beds on their own because a lot of force is required to adjust the angle of the back support.

For such reasons, in recent years, the electric medical beds enabling the patients to adjust the angle of the back support on their own are preferred even though the purchase cost is somewhat significant.

An example of such a conventional medical electric bed is disclosed in Korean Patent No. 10-0621349 (hereinafter referred to as a “related document”).

Referring to FIGS. 1 and 2 of the related document, in large, the conventional medical electric bed is configured to include: a bed frame 10; an operation module 20 positioned on an upper part of the bed frame 10 and including a back support unit 21 and a leg support unit 22, which are rotatably coupled to each other; a drive module 30 composed of a plurality of links rotatably coupled to the operation module 20 and driven by an external force; and a power module 40 positioned at a lower part of the bed frame 10 to provide the external force for the operation of the drive module 30.

The power module 40 is composed of a first actuator 41 and a second actuator 42. In each of the actuators, the first actuator 41 applies power to tilt the back support unit 21, and the second actuator 42 applies power to tilt the leg support unit 22.

In addition, the back support unit 21 is positioned on an upper surface of the bed frame 10, so that one end thereof is rotatably coupled to a fixing unit 50 provided in the bed frame 10, and the other end thereof is tilted upward by the drive module 30. One end of a thigh unit 22a is rotatably coupled to the other side of the fixing unit 50, one end of a shin unit 22b is rotatably coupled to the other end of the thigh unit 22a, and one end of a foot support unit 22c is rotatably coupled to the other end of the thigh unit 22a. As such, the leg support unit 22 is configured so that the leg support unit 22 is tilted upward by the drive module 30 coupled to the bed frame 10.

However, in the conventional medical electric bed configured as described above, as a mattress part thereof is divided into a back support unit, a waist unit, a joint unit, a leg support unit, and the like, and able to be driven up and down, posture modifications required for treatment and nursing activities such as sitting up a critically ill patient having difficulty in moving his or her body or lifting a leg of the patient are possible to some extent. However, for a patient lying in bed and unable to move, there are serious problems in that as specific body parts of the patient are pressed against a bed for a long period of time, blood circulation is disrupted and air cannot be in contact with the skin of the patient for the long period of time, leading to accompanied complications such as pressure ulcers and skin diseases.

### SUMMARY

The embodiment of the present disclosure was devised to solve the above problems, and an objective of the present disclosure is to provide a pressure ulcer prevention bed and a control method thereof using body pressure sensors, wherein a plurality of raising and lowering link modules (i.e., keyboards) in a multi-parallelogram link structure in which raising and lowering is independently controlled in the form of raising and lowering link modules are arranged in parallel adjacent to each other along a surface of a bed, a plurality of body pressure sensors is arranged at predetermined intervals on the keyboards, and then the raising and lowering movements of the keyboards are appropriately controlled according to measured values of the body pressure sensors, so that it is highly effective in preventing pressure ulcers, skin diseases, etc.

According to an exemplary embodiment of the present disclosure to achieve the above objective, there is provided a pressure ulcer prevention bed using body pressure sensors, the pressure ulcer prevention bed including: a plurality of raising and lowering link modules arranged in parallel adjacent to an upper surface of a bed support unit and whose raising and lowering is independently controlled by parallelogram links; the body pressure sensors provided in the respective raising and lowering link modules and constantly collecting body pressure sensing information for each part of a human body applied to each raising and lowering link module; a servo motor provided in the parallelogram links of each raising and lowering link module; and a motion control unit connected to the servo motor through wired and wireless communication and independently controlling each raising and lowering link module by using the body pressure sensing information individually transmitted from the body pressure sensors, wherein the body pressure sensors each consists of a plurality of unit body pressure sensors, and each unit body pressure sensor is arranged with adjacent unit body pressure sensors in a row at predetermined intervals along an upper surface of each raising and lowering link module.

In addition, according to the exemplary embodiment, each unit body pressure sensor may include: a sensing plate arranged with adjacent sensing plates in the row at the predetermined intervals along the upper surface of each raising and lowering link module; and a load cell formed in a plate shape and provided between the sensing plate and each raising and lowering link module.

In addition, according to the exemplary embodiment, each unit body pressure sensor may further include first spacers between the load cell and the upper surface of each raising and lowering link module, and as a body pressure is applied to an upper surface of the sensing plate, the body pressure sensing information may be measured within a thickness range of each first spacer while the load cell is elastically pressed.

In addition, according to the exemplary embodiment, between the sensing plate and the load cell, each unit body pressure sensor may further include: a second spacer provided between central parts of the sensing plate and the load cell, and configured to maintain a gap between the sensing plate and the load cell; a fastening screw for integrally coupling the central parts of the sensing plate, the second spacer, and the load cell to each other; and a silicone resin layer formed on an upper surface of the load cell to surround the second spacer and configured to provide elasticity, so as to allow the sensing plate to be elastically pressed or restored to an original position thereof.

In addition, according to the exemplary embodiment, the pressure ulcer prevention bed may further include: a user interface unit; a host interface unit; a multi-servo communication unit for connecting the servo motor provided in each raising and lowering link module through the wired and wireless communication; and a multi-sensor communication unit for connecting the body pressure sensors provided in the raising and lowering link modules through the wired and wireless communication.

In addition, according to the exemplary embodiment, each body pressure sensor may further include a body pressure sensing information processing unit for processing the body pressure sensing information.

In addition, according to the exemplary embodiment, the host interface unit may be connected to the multi-servo communication unit and the multi-sensor communication unit through Controller Arca Network (CAN) communication.

Meanwhile, according to an exemplary embodiment of the present disclosure, there is provided a control method of controlling a pressure ulcer prevention bed using body pressure sensors, the pressure ulcer prevention bed including: a plurality of raising and lowering link modules arranged in parallel adjacent to an upper surface of a bed support unit and whose raising and lowering is independently controlled by parallelogram links; the body pressure sensors provided in the respective raising and lowering link modules and constantly collecting body pressure sensing information for each part of a human body applied to each raising and lowering link module; a servo motor provided in the parallelogram links of each raising and lowering link module; and a motion control unit connected to the servo motor through wired and wireless communication and independently controlling each raising and lowering link module by using the body pressure sensing information individually transmitted from the body pressure sensors, the body pressure sensors each consisting of a plurality of unit body pressure sensors, and each unit body pressure sensor being arranged with adjacent unit body pressure sensors in a row at predetermined intervals along an upper surface of each raising and lowering link module, wherein, in a case where the body pressure sensing information collected from the body pressure sensors of the plurality of respective raising and lowering link modules is read by the motion control unit and body pressure sensing information transmitted from a body pressure sensor of a specific raising and lowering link module approaches 32 mmHg that is a pressure ulcer critical pressure at which a pressure ulcer is likely to occur, just before the specific raising and lowering link module with a pressure having come close to the pressure ulcer critical pressure reaches the pressure ulcer critical pressure, not only movements are stopped for a preset certain residence time after lowering only the specific raising and lowering link module with the pressure having come close to the pressure ulcer critical pressure from an initial first vertical position h1 where all the raising and lowering link modules comprising the specific raising and lowering link module are all parallel to a second vertical position h2 that is relatively lower than the first vertical position h1, but also the specific raising and lowering link module stopped at the second vertical position h1 is raised again to the initial first vertical position h1 after the certain residence time has elapsed, so that control is independently performed for the raising and lowering movements and vertical positions of each raising and lowering link module according to the body pressure sensing information, and alternatively, in a case of a failure where the body pressure sensing information collected from the body pressure sensors of each raising and lowering link module is read to independently control the movements and positions of the specific raising and lowering link module, duration control as a next step is automatically performed to repeatedly control the raising and lowering movements of each raising and lowering link module for a preset time.

In addition, according to the exemplary embodiment, in the motion control unit, the maximum body pressure may be calculated in each raising and lowering link module by Equation 1 below:

$${P}_{mi} = {max}_{i} {p}_{ij} , j = 1 , 2 , ⋯ , M \qquad ( Equation 1 )$$

(where, Pmi is the maximum pressure of a raising and lowering link module i, Pij is a body pressure measured at a j-th sensor of the i-th raising and lowering link module, and M is the number of body pressure sensors in each raising and lowering link module), an error ei may be calculated by Equation 2 below:

$${c}_{i} = min ( {p}_{\text{?}} - {p}_{mi} , 0 ) , i = 1 , 2 , … , N \qquad ( Equation 2 )$$

$$\text{?} \text{indicates text missing or illegible when filed}$$

(where, pₛᵥ is a target pressure value, pₘᵢ is the maximum pressure of the keyboard i, and uᵢ is a keyboard input), a total error eRMS may be calculated by Equation 3 below:

$${e}_{RMS} = \sqrt{\frac{{\sum }_{i = 1}^{N} {e}_{i}^{2}}{N}} , i = 1 , 2 , … , N \qquad ( Equation 3 )$$

(where, N is the number of raising and lowering link modules), and the motion control unit may independently control the raising and lowering operation and vertical positions of each raising and lowering link module so that a total error value calculated by the above Equations converges to zero.

In addition, according to the exemplary embodiment, the motion control unit may use a Proportional-Integral (PI) control method or an Integral-Proportional (IP) control method.

In addition, according to the exemplary embodiment, the duration control may divide the raising and lowering link modules into odd-numbered raising and lowering link modules and even-numbered raising and lowering link modules, and then repeatedly and alternately control the raising and lowering operation of the odd-numbered raising and lowering link modules and even-numbered raising and lowering link modules for a preset lowering time T1 and a preset raising time T2.

In addition, according to the exemplary embodiment, a sum of the lowering time T1 and the raising time T2 may be within 30 minutes.

As described above, the embodiment of the present disclosure has an excellent medical effect that a plurality of raising and lowering link modules whose raising and lowering are independently controlled is arranged in parallel adjacent to each other in the form of raising and lowering link modules along a surface of a bed, and the raising and lowering movements of the raising and lowering link modules are appropriately controlled according to a patient's treatment or nursing condition, thereby preventing serious side effects such as pressure ulcers and skin diseases, which occur as the patient's body is pressed against the bed for a long period of time, resulting in poor blood circulation or lack of contact with air.

### BRIEF DESCRIPTION OF THE DRAWINGS

FIG. 1 is a perspective view illustrating an medical electric medical bed as a whole according to the present disclosure.

FIG. 2 is an enlarged perspective view illustrating a main part of a raising and lowering link module according to an exemplary embodiment of the present disclosure.

FIG. 3 is an exploded perspective view of FIG. 2.

FIG. 4 is an enlarged perspective view illustrating a main part of a raising and lowering link module according to another exemplary embodiment of the present disclosure.

FIG. 5 is an exploded perspective view of FIG. 4.

FIGS. 6A to 6C are schematic state views for describing link motion of the raising and lowering link module of FIG. 4. FIG. 6A illustrates a normal motion state in a case of a single parallelogram link, FIG. 6B illustrates an abnormal motion state according to the occurrence of a change point in FIG. 6A, and FIG. 6C illustrates a normal motion state in which the occurrence of a change point is prevented by applying a double parallelogram link according to the present disclosure.

FIG. 7 is a schematic view illustrating a motion mechanism of a raising and lowering link module according to the present disclosure.

FIG. 8 is a block diagram illustrating a pressure ulcer prevention bed and a control method thereof using body pressure sensors of the present disclosure.

FIG. 9 is a block diagram illustrating a motion control unit in a Proportional-Integral (PI) control method according to the exemplary embodiment of the present disclosure.

FIG. 10 is a block diagram illustrating a motion control unit in an Integral-Proportional (IP) control method according to the exemplary embodiment of the present disclosure.

FIG. 11 is a graph illustrating experimental results of the motion control unit in the PI control method.

FIG. 12 is a graph illustrating experimental results of the motion control unit in the IP control method

FIG. 13 is a control block diagram illustrating a servo motor driver according to the present disclosure.

FIG. 14 is a flowchart illustrating a control method of controlling the pressure ulcer prevention bed according to the present disclosure.

FIG. 15 is a block diagram illustrating a structure of a fuzzy controller as part of a sensor controller according to the present disclosure.

FIG. 16 is a flowchart illustrating duration control according to the exemplary embodiment of the present disclosure.

FIG. 17 is a plan view illustrating a use state of the pressure ulcer prevention bed using the body pressure sensors of the present disclosure.

FIGS. 18A to 18C is an exemplary view illustrating a body pressure distribution map according to the present disclosure.

FIG. 19 is an enlarged perspective view illustrating a main part of a raising and lowering link module according to a yet another exemplary embodiment of the present disclosure.

FIG. 20 is an exploded perspective view of FIG. 19.

FIG. 21 is an enlarged view of a main part of FIG. 20.

FIG. 22 is a bottom perspective view illustrating a body pressure sensor of FIG. 21.

FIG. 23 is an exploded perspective view of FIG. 22.

### DETAILED DESCRIPTION

The terms used herein are for the purpose of describing particular exemplary embodiments only and are not intended to be limiting the present disclosure. As used herein, the singular forms are intended to include the plural forms as well, unless the context clearly indicates otherwise. In the present specification, it will be understood that the terms “comprise”, “include”, “have”, etc., when used in this specification, specify the presence of stated features, integers, steps, operations, elements, components, and/or combinations thereof, but do not preclude the presence or addition of one or more other features, integers, steps, operations, elements, components, and/or combinations thereof.

Unless otherwise defined, all terms including technical and scientific terms used herein have the same meaning as commonly understood by those skilled in the art to which the present disclosure belongs.

It will be further understood that terms as defined in dictionaries commonly used herein should be interpreted as having meanings that are consistent with their meanings in the context of the relevant art and should not be interpreted in an idealized or overly formal sense unless expressly so defined in the present specification.

Hereinafter, configurations and operational relationships of a medical electric bed of the present disclosure will be described in detail with reference to the attached drawings.

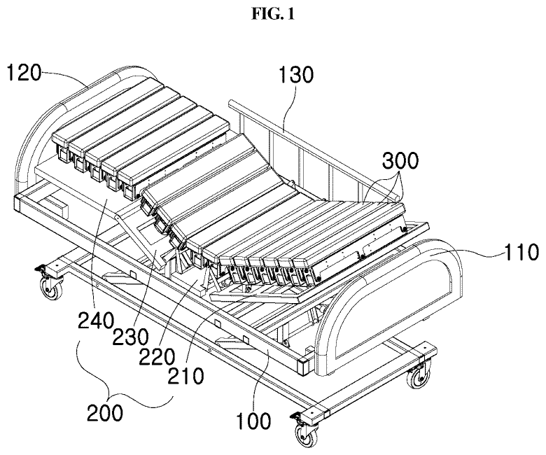

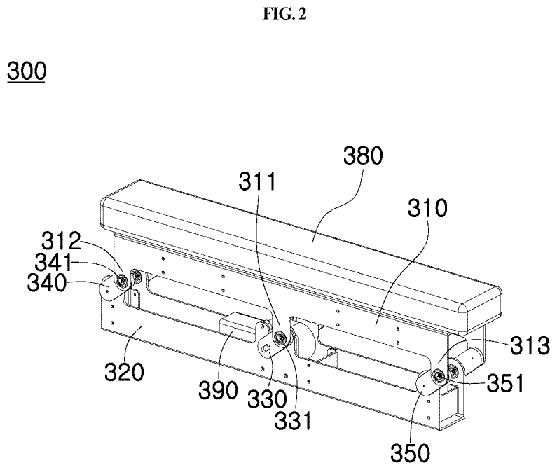

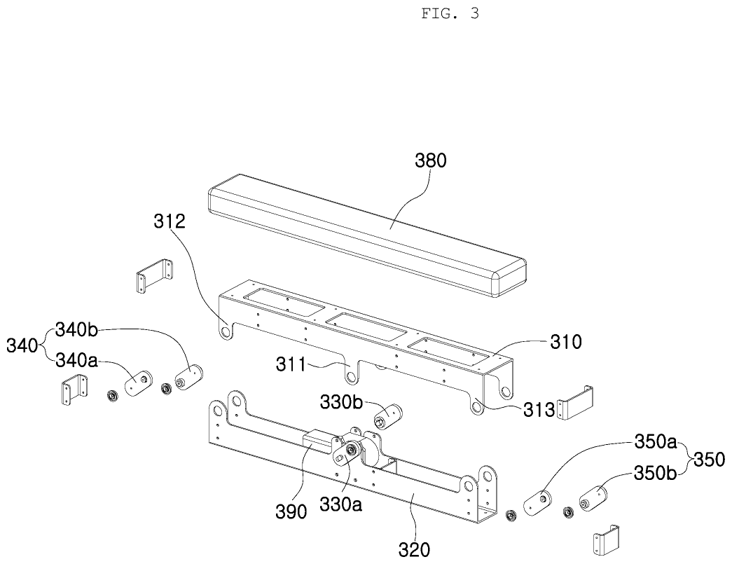

FIG. 1 is a perspective view illustrating a medical electric bed as a whole according to the present disclosure. FIG. 2 is an enlarged perspective view illustrating a main part of a raising and lowering link module according to an exemplary embodiment of the present disclosure. FIG. 3 is an exploded perspective view of FIG. 2.

Hereinafter, a configuration of the medical electric bed according to the exemplary embodiment of the present disclosure will be described with reference to FIGS. 1 to 3.

First, the medical electric bed of the present disclosure is provided with a support frame 200 capable of supporting each part of a human body on an upper part of a bed frame 100, which is usually made of steel structure. The support frame 200 is divided into a back support unit 210, a waist support unit 220, a knee support unit 230, a leg support unit 240, etc., and raising and lowering or a rotation angle of each of the support units 210, 220, 230, and 240 may be selectively adjusted.

For example, when a patient with limited mobility is seated, the back support unit 210 may be pushed and raised upward. Meanwhile, a patient with impaired legs may control rotation angles or raising and lowering, such as raising the leg support unit 240 upward.

In addition, the medical electric bed is provided with a headboard 110 and a footboard 120 respectively on a head side and a leg side of a patient on the bed frame 100, and is further provided with a safety bar 130, which goes up and down and is fixed, on a side of the bed frame 100 in order to prevent the patient from rolling and falling off the bed.

Meanwhile, as a characteristic component of the present disclosure, on an upper surface of each of the support units 210, 220, 230, and 240 of the support frame 200, a plurality of raising and lowering link modules 300 whose raising and lowering are independently controlled by parallelogram links, is arranged in parallel adjacent to each other, and a servo motor 390 is coupled to one of link coupling shafts of each raising and lowering link module 300 for link operation.

In this case, it is understandable that each raising and lowering link module 300 is provided with a separate servo motor 390 so that the raising and lowering of each raising and lowering link module 300 are independently controlled by the servo motor 390.

Hereinafter, referring to FIGS. 2 and 3, according to the exemplary embodiment, each raising and lowering link module 300 has an overall shape of two parallelogram link structures connected to each other and arranged side by side on a horizontal line, wherein a phase offset link piece 330 is disposed between the two parallelogram links, and the two parallelogram link structures have a structure of sharing the phase offset link piece 330 with each other. In this case, a vertical position of a link coupling shaft 331 of the phase offset link piece 330 is positioned downward lower than coupling shafts 341 and 351 of other links on the left and right.

More specifically, each of the raising and lowering link modules 300 is configured to include: an upper link frame 310 having a predetermined length from side to side and provided with a plurality of coupling ribs 311, 312, and 313, which are formed to protrude integrally and for link coupling downwards; and a lower link frame 320 having a structure opposite to the upper link frame 310 as a whole, and being disposed below the upper link frame 310 and being link-coupled thereto.

The longitudinal cross-sectional shapes of the upper link frame 310 and the lower link frame 320 in the direction perpendicular to a longitudinal direction thereof respectively have the shapes of “ [symbol] ” and “ [symbol] ” and accordingly, a servo motor is installable at a position inside a cross section of the lower link frame 320.

In this case, as shown in FIGS. 2 and 3, the lower link frame 320 may be provided with coupling ribs (no reference numerals) in the same shapes as the coupling ribs 311, 312, and 313 of the upper link frame 310, or may perform the same function as a parallelogram link even though only coupling holes (no reference numeral) are formed instead of the coupling ribs (no reference numerals) in the lower link frame 320, and thus, a detailed description for the structure of the lower link frame 320 will be omitted.

In addition, a plurality of link pieces 330, 340, and 350 is coupled respectively between the coupling ribs 311, 312, and 313 of the upper and lower link frames 310 and 320. First, a phase offset link piece 330 in which a vertical position of a coupling shaft 331 is positioned lower than those of the other coupling shafts 341 and 351 adjacent to the left and right of the coupling shaft 331 is coupled to the coupling rib 311 in the middle.

In addition, a first link piece 340 is coupled to the coupling rib 312 formed at a left end relative to the phase offset link piece 330, and a second link piece 350 is hinge coupled to the coupling rib 313 at a right end relative to the phase offset link piece 330. In this case, respective coupling shafts 341 and 351 of the first link piece 340 and the second link piece 350 are positioned higher than the coupling shaft 331 of the phase offset link piece 330 described above.

In addition, all the link pieces 330, 340, and 350, i.e., phase offset link pieces 330a and 330b, first link pieces 340a and 340b, and second link pieces 350a and 350b, are provided separately one on a side and the other on an opposite side at predetermined intervals, so as to form a pair of two each. Accordingly, when performing link motion from side to side on the lower link frame 320, the upper link frame 310 is stably supported without shaking towards a side perpendicular to a left and right movement direction of the links, and is enabled to perform the link motion.

In this case, as described above, among the coupling ribs 311, 312, and 313 of the upper link frame 310, the coupling rib 311 in the middle thereof to which the phase offset link piece 330 is coupled is formed to be longer in length downward than those of the other coupling ribs 312 and 313. Accordingly, a vertical position of the coupling shaft 331 positioned in the middle is positioned lower than the other coupling shafts 341 and 351 on the left and right.

In addition, as described above, a servo motor 390 is installed in the lower link frame 320, and a rotation shaft of the servo motor 390 is coupled to the phase offset link piece 330, thereby functioning as a drive shaft. Therefore, as the phase offset link piece 330 is driven from side to side by the electric force of the servo motor 390, the first link piece 340 and the second link piece 350, which are link-coupled thereto, are also simultaneously driven together from side to side.

In addition, a bed piece 380 for supporting the load of a user's body is further provided on an upper surface of each upper link frame 310. The bed piece 380 may be, for example, a cushion material in the form of a sponge with a waterproof coating finished on outer sides thereof, or may also be realized with a hard wood material without a cushion.

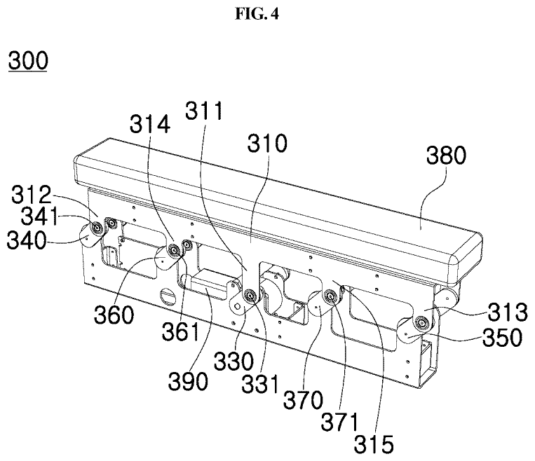

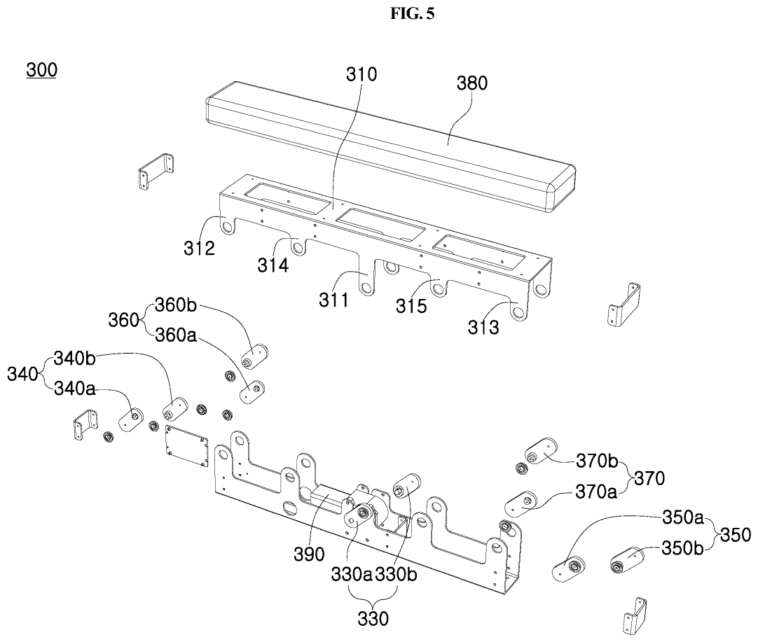

Meanwhile, FIG. 4 is an enlarged perspective view illustrating a main part of a raising and lowering link module according to another exemplary embodiment of the present disclosure. FIG. 5 is an exploded perspective view of FIG. 4. FIGS. 4 and 5 are views illustrating another exemplary embodiment of FIGS. 2 and 3 described above, and illustrate that each raising and lowering link module of the present disclosure is implemented in the form of a quadruple parallelogram link.

More specifically, the quadruple parallelogram link is a structure in which three links are shared among each parallelogram link, and has a configuration in which a third link piece 360 and a fourth link piece 370 are added to the structure of the double parallelogram link described above. Accordingly, the upper link frame 310 is further provided with coupling ribs 314 and 315 and coupling shafts 361 and 371 for link coupling the third and fourth link pieces 360 and 370, respectively.

Therefore, as described in the exemplary embodiment, the phase offset link piece 330 is driven according to the driving of the servo motor 390 coupled to the phase offset link piece 330, and accordingly, the first to fourth link pieces 340, 350, 360, and 370, which are link-coupled to the phase offset link piece 330, are also moved simultaneously.

Since the quadruple parallelogram link structure is supported by more link pieces than the double parallelogram link structure, it is a structure with higher stability in terms of load support and operational reliability.

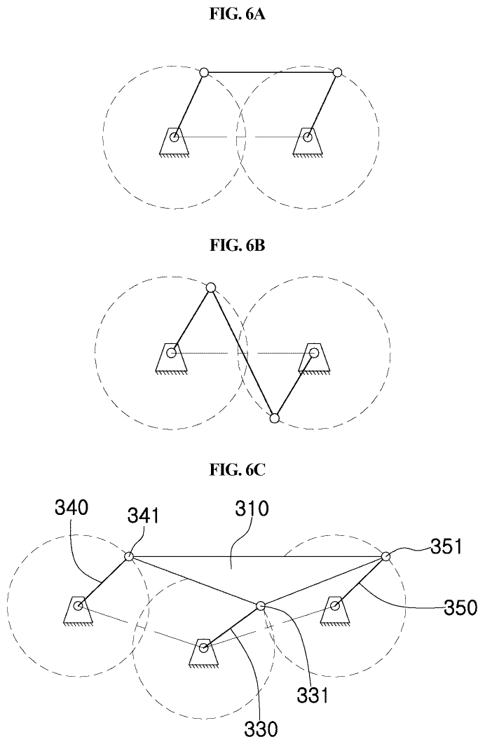

FIGS. 6A to 6C are schematic state views for describing link motion of the raising and lowering link module of FIG. 4. FIG. 6A illustrates a normal motion state in a case of a single parallelogram link, FIG. 6B illustrates an abnormal motion state according to the occurrence of a change point in FIG. 6A, and FIG. 6C illustrates a normal motion state in which the occurrence of a change point is prevented by applying a double parallelogram link according to the present disclosure.

Hereinafter, when the link motion of each raising and lowering link module according to the present disclosure are described with reference to FIGS. 6A to 6C, firstly, the attached FIG. 6A illustrates a case of a single parallelogram link to which the present disclosure is not applied. In this case, in a normal motion state, each link maintains the overall structure of a parallelogram link and mostly performs normal motion.

However, as shown in FIG. 6B, the single parallelogram link structure in FIG. 6A may occasionally have a change point problem in which a driven link does not properly follow the one-way motion of a drive link.

Here, the change point is related to a phenomenon in which all links are placed in a straight line as shown in FIG. 6B. In a case where such a change point occurs, the motion of an output link becomes unpredictable. In this case, unlike the normal motion in FIG. 6A, the change point causes an abnormal motion generating rattling shocks and vibrations when a link motion is performed.

Accordingly, in order to prevent the occurrence of such a change point at the source, each link in the raising and lowering link module according to the present disclosure is proposed to use a multiple parallelogram link in the form of at least two or more parallelogram links connected and installed.

As an exemplary embodiment of such a multiple parallelogram link, the double parallelogram link as previously shown in FIGS. 2 and 3 may be applied, and an example of which is schematically shown in FIG. 6C.

The double parallelogram link shown in FIG. 6C has a form of the shared link positioned in the very middle, i.e., the phase offset link in which the coupling shaft 331 of the phase offset link piece 330 is positioned lower on a horizontal line than the coupling shafts 341 and 351 of the other links on the left and right.

Therefore, during the circular motion of the double parallelogram link, the occurrence of a change point is prevented by the phase offset link piece 330, thereby having an effect of preventing the occurrence of an abnormal motion.

Meanwhile, in a state of the phase offset link piece 330 being stood vertically, the upper link frame 310 is in a state of being positioned at the highest point, and at this time, the raising and lowering link module 300 is in a raised state.

In contrast, in a state where the phase offset link piece 330 is rotated from side to side, the upper link frame 310 is in a state of being positioned at the lowest point, and accordingly, the raising and lowering link module 300 is in a lowered state.

Therefore, the raising and lowering of each raising and lowering link module 300 becomes independently controllable as described above, and accordingly, the shapes of the upper surfaces of the upper link frame 310 and the bed piece 380 of each raising and lowering link module 300 may be changed so as to form flat surfaces side by side on a horizontal plane or to form convex and concave surfaces.

Accordingly, in a case of a patient who has to lie in bed for a long time, a physical condition should be changed on a cyclic basis or by situation. The embodiment of the present disclosure independently controls the raising or lowering movement of each raising and lowering link module, thereby preventing serious side effects such as pressure ulcers caused by specific body parts of a patient constantly pressed by the body's weight, impeding blood circulation or preventing air from reaching the skin.

Meanwhile, previously, in FIG. 6C, only the case of the double parallelogram link is illustrated, but the multiple parallelogram link is also applicable without limit. For example, as in another exemplary embodiment of the present disclosure illustrated in FIGS. 4 and 5 described above, the quadruple parallelogram link is applicable, and in this case, not only the load is supported more firmly than the double parallelogram link, but also the occurrence of a change point is suppressed more clearly, whereby an effect of improving the reliability of movements may be achieved.

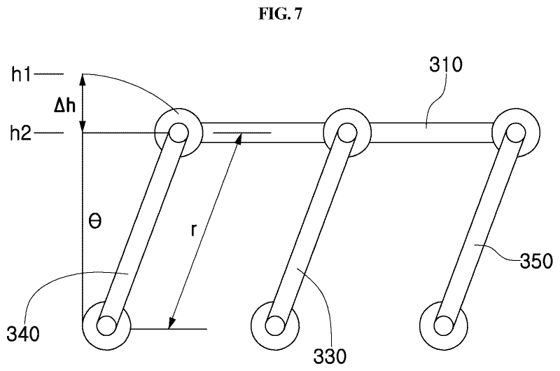

FIG. 7 is a schematic view illustrating a motion mechanism of a raising and lowering link module according to the present disclosure.

Hereinafter, when the motion mechanism of the raising and lowering link module 300 according to the present disclosure is described with reference to FIG. 7, a servo motor 390 receives an input with a rotation angle, and a height value h of an upper link frame 310 is determined according to lengths r of link pieces 330, 340, and 350 connected to the servo motor 390.

In addition, a required rotation angle of the servo motor 390 may be obtained by substituting a desired movement height into Equations 1 and 2 below.

$$h = r ( 1 - cos θ ) \qquad ( Equation 1 )$$

$$θ = arccos ( 1 - \frac{h}{r} ) \qquad ( Equation 2 )$$

Here, θ is a rotation angle of the raising and lowering link module 300, r is a rotation radius of the raising and lowering link module 300, and h is a height (i.e., a vertical position) of the raising and lowering link module 300. h1 is an initial vertical position where each raising and lowering link module 300 is arranged in parallel, and h2 is a vertical position in a state when the raising and lowering link module 300 is lowered by Δh.

In the pressure ulcer prevention bed and the control method thereof using the body pressure sensors of the present disclosure, the plurality of raising and lowering link modules 300 of which the raising and lowering are independently controlled by the parallelogram links is arranged in parallel adjacent to each other on the upper surface of the bed support unit, and the movements of each raising and lowering link module is controlled by the servo motor 390.

To this end, first, body pressure sensing information for each part of a human body applied to each raising and lowering link module 300 is constantly collected and transmitted to the motion control unit through the body pressure sensor 1510 provided in each raising and lowering link module 300.

Next, in a case where the body pressure sensing information collected from the body pressure sensors of the plurality of respective raising and lowering link modules is read by the motion control unit and body pressure sensing information transmitted from a body pressure sensor of a specific raising and lowering link module approaches 32 mmHg that is a pressure ulcer critical pressure at which a pressure ulcer is likely to occur, just before the specific raising and lowering link module with a pressure having come close to the pressure ulcer critical pressure reaches the pressure ulcer critical pressure, not only movements are stopped for a preset certain residence time after lowering only the specific raising and lowering link module with the pressure having come close to the pressure ulcer critical pressure from an initial first vertical position (h1) where all the raising and lowering link modules comprising the specific raising and lowering link module are all parallel to a second vertical position (h2) that is relatively lower than the first vertical position (h1).

Next, the specific raising and lowering link module stopped at the second vertical position (h1) is raised again to the initial first vertical position (h1) after the certain residence time has elapsed, so that control is independently performed for the raising and lowering movements and vertical positions of each raising and lowering link module according to the body pressure sensing information.

For example, the certain residence time may be set to five seconds (sec) or more in consideration of the time when the raising and lowering link module 300 is lowered and the time when body pressure stabilizes after the lowering.

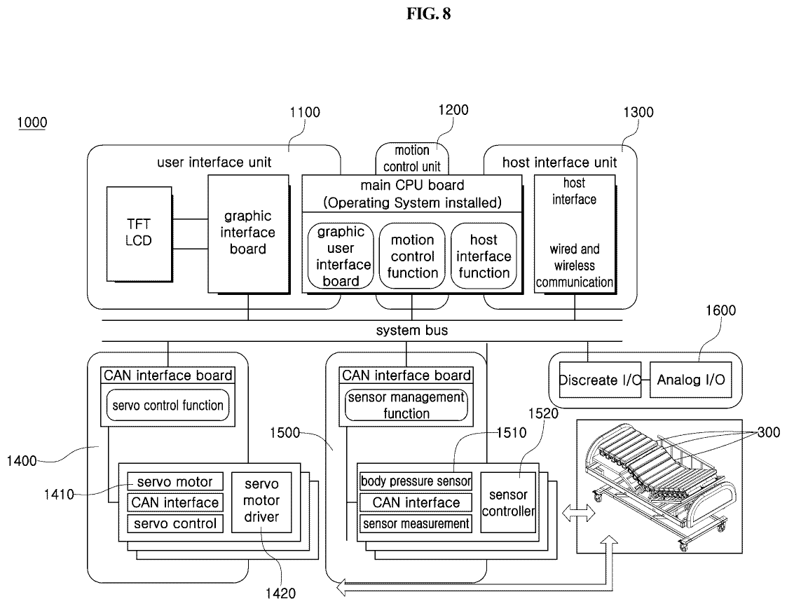

FIG. 8 is a block diagram illustrating the pressure ulcer prevention bed and the control method thereof using the body pressure sensors of the present disclosure.

A control system 1000 of the present disclosure may be configured to include: a user interface unit 1100; a motion control unit 1200; a host interface unit 1300; a multi-servo communication unit 1400 for performing connection of a servo motor 1410 provided in each raising and lowering link module 300 through wired and wireless communication; and a multi-sensor communication unit 1500 for connecting a body pressure sensor 1510 and a sensor controller 1520 to each other, which are provided in each raising and lowering link module 300 through the wired and wireless communication.

In addition, the control system 1000 may further include a sensor signal processing unit 1600 for processing body pressure sensor signals of each body pressure sensor 1510.

According to the exemplary embodiment, the host interface unit 1100, the multi-servo communication unit 1400, and the multi-sensor communication unit 1500 may be connected to each other through Controller Area Network (CAN) communication.

More specifically, the control system 1000 of the present disclosure may be configured to include 16 servo motor drivers 1420 for driving 16 raising and lowering link modules 300, a user interface unit 1100, a motion control unit 1200, and a host interface unit 1300, which are connected to each other through Controller Arca Network (CAN) communication.

In this case, the user interface unit 1100 may be a teach pendant for indicating bed operation modes.

In addition, the Controller Area Network (CAN) communication is a standard communication standard designed to enable microcontrollers or devices to communicate with each other in a vehicle without a host computer.

According to the exemplary embodiment, the servo motors 1410 of the present disclosure may use 16 three-phase BLDC motors, and the servo motor drivers 1420 for driving respective servo motors 1410 up and down may use a driver with square-wave drive type using Hall sensor signals.

For example, a servo motor driver 1420 may be mounted on each raising and lowering link module 300, and the host interface unit 1300 for communicating with each servo motor driver 1420 may include two CAN communication ports and one RS485 communication port.

In addition, each servo motor driver 1420 is capable of position control or speed control, and a position control mode and a speed control mode may be set in real time with CAN messages. In addition, ID recognition of the 16 servo motor drivers 1420 may be set sequentially by using the input/output (I/O) of each servo motor driver 1420.

Here, since each BLDC servo motor 1410 is capable of performing sufficient position control only with signal information from a Hall sensor, an algorithm for calculating position information with the three-phase Hall sensor is loaded in the control system of the present disclosure, and the hardware of each servo motor driver 1420 may be configured to provide support for CAN communication and RS485 communication.

The motion control unit 1200 transmits commands of position control and speed control to corresponding servo motor drivers 1420 through the CAN communication of the host interface unit 1300, and performs control of raising and lowering movements and vertical positions for respective servo motors 1410 by the servo motor drivers 1420.

According to the exemplary embodiment, a body pressure sensor 1510 may apply an FSR type thin film body pressure sensor. The body pressure sensor 1510 is mounted on each raising and lowering link module, and M body pressure sensors 1510 are connected to the sensor controllers 1520. Then, the N sensor controllers 1520 transmit NM pieces of body pressure sensing information to the motion control unit 1200 through the CAN communication, and the larger the NM (i.e., the greater the number of NMs), the greater the resolution of a body pressure sensing map.

However, it is preferable that the number of N and the number of M are limited to appropriate numbers for physical and practical reasons, and each body pressure sensor may be configured to be calibrated before use and to transmit body pressure sensing information, which is in a range of 0 to 80 mmHg, to the motion control unit.

Accordingly, when a control command is transmitted from the motion control unit 1200 to a servo motor driver 1420 installed in each of the 16 raising and lowering link modules 300 through the CAN communication, the servo motor driver 1420 controls each raising and lowering link module 300 to be driven up and down independently for pressure ulcer prevention.

In the present disclosure, as a motion control unit to independently control the raising and lowering movements and vertical positions of such servo motors, motion control units in a PI control method and an IP control method may be used.

The PI control method generates an output that is proportional to errors between a command value and measured values and an integral value of the errors, whereas the IP control method generates an output proportional to measured values and an integral value of errors between a command value and the measured values.

In the case of an IP controller, since there is no zero point in a transfer function, it is easy to set control gains, so as to allow overshoot, stabilization time, and the like to become desired characteristics, and when the same size of overshoot is used as a basis, a gain may be made larger than that of the PI control method. That is, a bandwidth may be increased, resulting in better response characteristics with respect to disturbance.

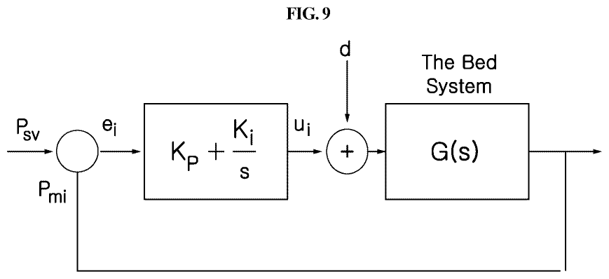

FIG. 9 is a block diagram illustrating a motion control unit in a Proportional-Integral (PI) control method according to the exemplary embodiment of the present disclosure.

A vertical position movement amount (Δh, see FIG. 7) of a raising and lowering link module may be modeled as being adjusted for a difference between a current body pressure and a critical body pressure (32 mmHg).

When the motion control unit in the PI control method is assumed to be a first-order system and expressed as a closed loop transfer function, it is as shown in Equation 3 below.

$$G ( s ) = \frac{b}{s + a} \qquad ( Equation 3 )$$

$${G}_{PI} ( s ) = \frac{{p}_{mi}}{{p}_{ω}} = \frac{b ( {k}_{\text{?}} s + {k}_{\text{?}} )}{{s}^{2} + ( a + {bk}_{\text{?}} ) s + {bk}_{\text{?}}} \qquad ( Equation 4 )$$

$$\text{?} \text{indicates text missing or illegible when filed}$$

Here, Pmi is the maximum pressure of an i-th raising and lowering link module, kp is a proportional term, and ki is an integral term.

In the case of the motion control unit in the PI control method expressed in Equation 4 above, a zero point exists in s₂=−kᵢ/kₚ, and the size of overshoot increases due to the influence of this zero point. Therefore, when the motion control unit in the IP control method is used, a problem of overshoot response due to such a zero point in a PI control system may be solved.

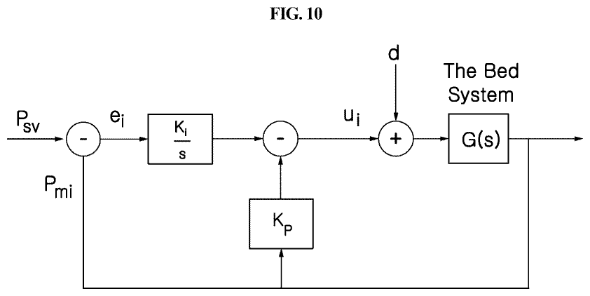

FIG. 10 is a block diagram illustrating a motion control unit in an Integral-Proportional (IP) control method according to another exemplary embodiment of the present disclosure.

Unlike the motion control unit in the PI control method in which an error e of body pressure is proportionally controlled, the motion control unit in the IP control method performs proportional control on body pressure sensing information that is output.

When the block diagram of FIG. 10 is expressed as a transfer function, it is as shown in Equation 5 below.

$${G}_{IP} ( s ) = \frac{{p}_{mi}}{{p}_{\text{?}}} = \frac{{bk}_{\text{?}}}{{s}^{2} + ( a + {bk}_{p} ) s + b \text{?}} \qquad ( Equation 5 )$$

$$\text{?} \text{indicates text missing or illegible when filed}$$

In the motion control unit in the IP control method in Equation 5, a zero point of the PI control method is removed, whereby control is enabled to reduce the overshoot.

### Control Performance Experimental Example of PI Control Method and IP Control Method

In the present experimental example, the experiment was conducted by separately applying motion control units 1200 in the PI control method and the IP control method in order to control an error e between a body pressure applied to each raising and lowering link module 300 and a pressure ulcer critical pressure. A height (h, a vertical position) of each raising and lowering link module 300 was controlled by the motion control units 1200 in the PI or IP control method.

In addition, the maximum body pressure is calculated in each raising and lowering link module by Equation 6 below:

$${p}_{mi} = {max}_{j} {p}_{ij} , j = 1 , 2 , … , M \qquad ( Equation 6 )$$

Here, Pmi is the maximum pressure of an i-th raising and lowering link module, Pij is a body pressure measured at a j-th sensor of the i-th raising and lowering link module, and M is the number of body pressure sensors in each raising and lowering link module.

In this case, when a maximum body pressure value exceeds 32 mmHg, it means that the maximum body pressure value exceeds the pressure ulcer critical pressure, whereby an actual pressure ulcer occurs when the body pressure lasts for a predetermined period of time.

In addition, an error ei may be calculated by Equation 7 below.

$${e}_{i} = min ( {p}_{\text{?}} - {p}_{mi} , 0 ) , i = 1 , 2 , … , N \qquad ( Equation 7 )$$

$$\text{?} \text{indicates text missing or illegible when filed}$$

Here, p_{zo} is a target pressure value, pₘᵢ is the maximum pressure of the keyboard i, and uᵢ is a keyboard input. In this case, the total error is calculated as follows.

In addition, the total error may be calculated by Equation 8 below.

$${e}_{RMS} = \sqrt{\frac{{\sum }_{i = 1}^{N} {e}_{i}^{2}}{N}} , i = 1 , 2 , … , N \qquad ( Equation 8 )$$

N is the number of raising and lowering link modules.

A height (h, a vertical position) of a raising and lowering link module was adjusted according to the above Equation, and the characteristics of e_{RMS} obtained when a change in a body pressure value occurs for a changed height were compared and reviewed with each other.

Through the above-described process, in the present experiment, an experiment objective was set to have the total error to converge to zero by applying the motion control unit of the PI or IP control method to adjust the height of each raising and lowering link module, so as to remove the error in each raising and lowering link module.

As a raising and lowering link module is lowered, a body pressure applied to the corresponding raising and lowering link module also decreases. However, in an instance where the raising and lowering link module is lowered rapidly, this instance may cause strain to a patient, so it is preferable to control height adjustment in a way of reducing a height difference between the corresponding raising and lowering link module and surrounding raising and lowering link modules.

In the present experiment, results were compared and examined for the fact that each raising and lowering link module was lowered in stages, and the total error becomes zero with minimal movements.

For example, considering the time when a raising and lowering link module is lowered and the time when body pressure stabilizes after the lowering, a timer period of the motion control unit was set to greater than or equal to five seconds (sec).

The parameter values of the motion control unit of the PI control method used in the experiment are shown in Table 1 below.

TABLE 1

| K [symbol] | K [symbol] | P [symbol] (mmHg) |
|---|---|---|
| 0.5 | 0 | 32 |
| 0.5 | 1 | 32 |

[symbol]

indicates data missing or illegible when filed

In the experiment on the motion control unit of the PI control method, body pressure control was tested about a supine posture on a pressure ulcer prevention bed equipped with body pressure sensors for an adult male weighing 175 cm and 88 kg. The experiment was conducted by dividing test cases into a case with only a proportional term (i.e., P control) and a case with a proportional-integral term (i.e., PI control).

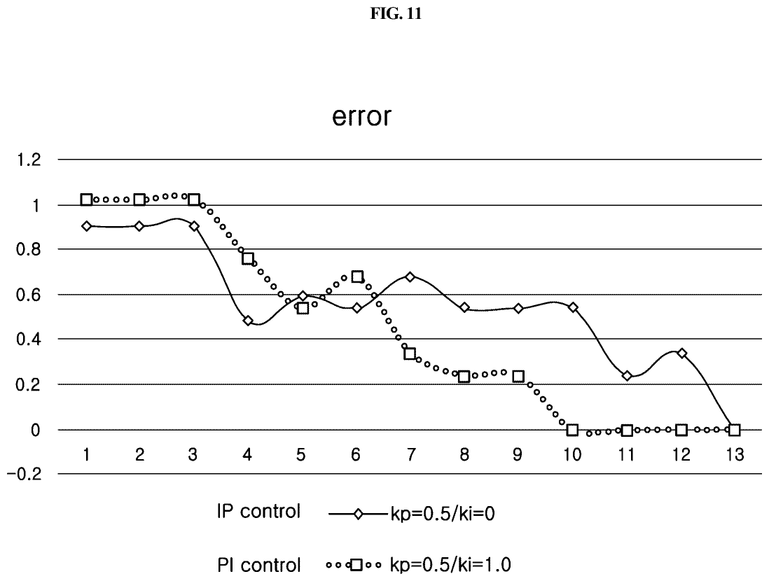

FIG. 11 is a graph illustrating experimental results of the motion control unit in the PI control method.

As shown in FIG. 11, it may be confirmed that the total error converges to zero more quickly because the PI control in the case of performing integral (I) control reduces residual deviation.

Table 2 below is an experiment result table summarizing the numerical data of the PI control method. (In the following, a keyboard refers to a raising and lowering link module).

TABLE 2

| | | Maximum | | Maximum | |
|---|---|---|---|---|---|
| | | body | | body | |
| | Error | pressure | Error | pressure | |
| | before | before | after | after | Keyboard |
| Keyboard | control | control | control | control | height |
| number | (mmHg) | (mmHg) | (mmHg) | (mmHg) | (mm) |
| 1 | 0 | 0 | 0 | 0 | 0 |
| 2 | 0 | 0 | 0 | 0 | 0 |
| 3 | 0 | 13 | 0 | 14 | 0 |
| 4 | 0 | 0 | 0 | 5 | 0 |
| 5 | 0 | 17 | 0 | 15 | 0 |
| 6 | −5 | 37 | 0 | 20 | −4 |
| 7 | 0 | 0 | 0 | 0 | 0 |
| 8 | 0 | 0 | 0 | 4 | 0 |
| 9 | 0 | 28 | 0 | 16 | 0 |
| 10 | −5 | 37 | 0 | 21 | −3 |
| 11 | 0 | 0 | 0 | 0 | 0 |
| 12 | 0 | 20 | 0 | 16 | 0 |
| 13 | 0 | 23 | 0 | 14 | 0 |
| 14 | −7 | 39 | 0 | 21 | −18 |
| 15 | −5 | 37 | 0 | 20 | −5 |
| 16 | 0 | 0 | 0 | 0 | 0 |
| 17 | −2 | 34 | 0 | 17 | −14 |

Meanwhile, the parameter values of the motion control unit in the IP control method used in the experiment are shown in Table 3 below.

TABLE 3

| K [symbol] | K [symbol] | P [symbol] (mmHg) |
|---|---|---|
| 0.3 | 0.3 | 32 |
| 0.3 | 1.2 | 32 |

[symbol]

indicates data missing or illegible when filed

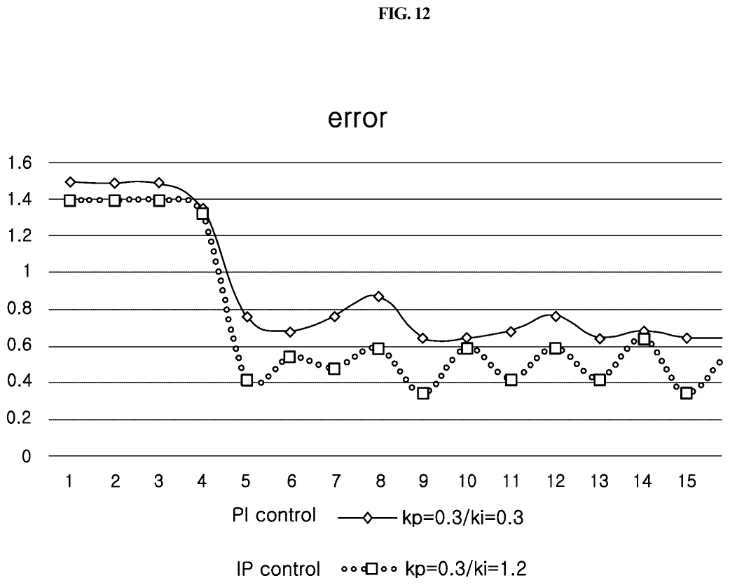

Meanwhile, FIG. 12 is a graph illustrating experimental results of the motion control unit in the IP control method

When the experimental results between the PI and IP control methods above are compared to each other, it may be confirmed that the PI control method proportionally controls the error from a pressure ulcer critical pressure value, so a convergence time to zero is faster than that of the IP control method.

In contrast, it was observed that despite stably converging an error to zero, the IP control method takes time slower than that of the PI control method, and since the raising and lowering link module continues to vibrate during the IP control, there exists a possibility that a patient may not feel comfortable.

Accordingly, as described above, body pressure sensors are installed on each raising and lowering link module of a pressure ulcer prevention bed configured with a plurality of raising and lowering link modules, and then the above-described raising and lowering link modules are divided into modules in the PI control method and modules in the IP control method according to body pressure sensing information, thereby separately performing the control performance experiments of the motion control units.

To this end, through the method of measuring a body pressure for each part of the body by each body pressure sensor, controlling the body pressure through feedback, and then controlling the body pressure detected by each raising and lowering link module to be within the pressure ulcer threshold pressure (32 mmHg), the respective numbers of movements of each raising and lowering link module is compared and reviewed until each error occurred in the PI control method and IP control method converges to zero, whereby it was confirmed that in the pressure ulcer prevention bed according to the present disclosure for removing a pressure ulcer, the PI control method is more advantageous than the IP control method in terms of pressure control speed and movement amounts of each raising and lowering link module.

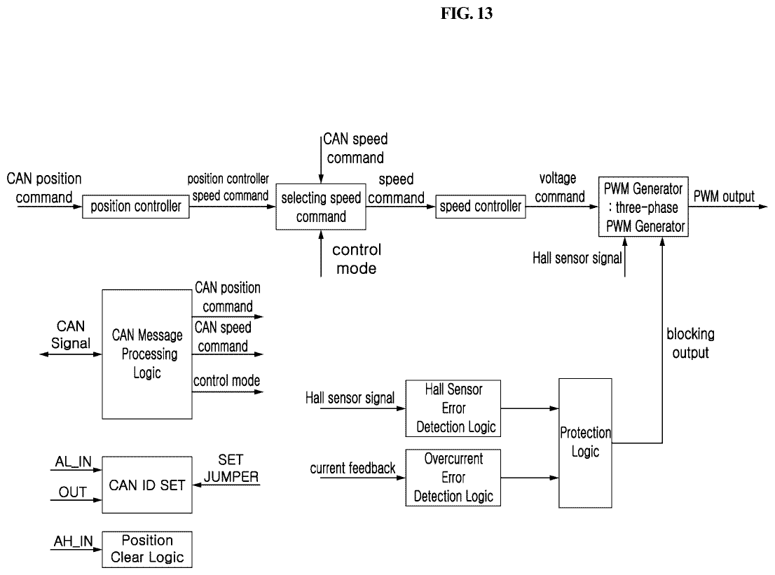

FIG. 13 is a control block diagram illustrating a servo motor driver according to the present disclosure.

Accordingly, when control commands are transmitted from the motion control unit 1200 to servo motor drivers 1420 respectively installed in the 16 raising and lowering link modules 300 through the CAN communication, the servo motor drivers 1420 control the respective raising and lowering link modules 300 to be driven up and down independently for pressure ulcer prevention.

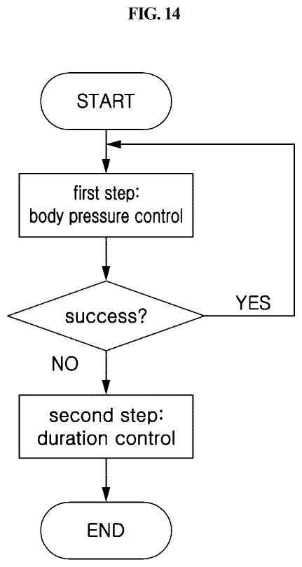

FIG. 14 is a flowchart illustrating a control method of controlling a pressure ulcer prevention bed according to the present disclosure.

The control method of the present disclosure may be divided into a first step of body pressure control and a second step of duration control.

For example, the body pressure control in the first step may be performed by a fuzzy logic system (FLS) programmed in a motion control unit.

The fuzzy logic system (FLS) may define an input data set as a non-linear mapping to scalar output data. The fuzzy logic system may consist of four main parts: fuzzification, rule verification, inference, and defuzzification.

A fuzzy logic process of the above fuzzy logic system is as follows.

First, a clear input data set is collected and transformed into a fuzzy set by using fuzzy language variables, fuzzy language terms, and membership functions (this step is known as the fuzzification).

Thereafter, the inference is performed on the basis of a set of rules.

Finally, a fuzzy output value is determined by using membership functions in the stage of the defuzzification.

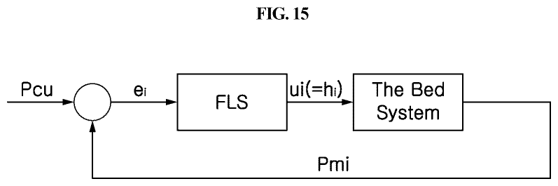

In this case, a fuzzy controller is used as a sensor controller 1520 for controlling an error between a pressure applied to each raising and lowering link module 300 and a pressure ulcer critical pressure, and a structure of the fuzzy controller is shown in FIG. 15.

FIG. 15 is a block diagram illustrating a structure of a fuzzy controller as part of a sensor controller according to the present disclosure.

Referring to FIG. 15, a body pressure sensor 1510 is mounted on each raising and lowering link module 300, and a height of each raising and lowering link module 300 may be controlled by the fuzzy controller.

Here, Pcu is a pressure ulcer critical pressure, Pmi is the maximum pressure of a raising and lowering link module i, hi is a height of the raising and lowering link module i, ei is a difference between Pcu and Pmi (Pcu−Pmi), and ui (=hi) is a movement amount of the raising and lowering link module.

In addition, an error ei may be calculated by Equation 9 below.

$${e}_{i} = min ( {p}_{mi} - {p}_{cu} , 0 ) , i = 1 , 2 , … N \qquad ( Equation 9 )$$

In addition, the total error may be calculated by Equation 10 below.

$${e}_{RMS} = \sqrt{\frac{{\sum }_{i = 1}^{N} {{e}_{i}}^{2}}{N}} , i = 1 , 2 , … N \qquad ( Equation 10 )$$

N is the number of raising and lowering link modules.

In addition, the maximum pressure Pₘᵢ of an i-th raising and lowering module may be obtained by Equation 11 below.

$${P}_{mi} = max {p}_{ij} , j = 1 , 2 , … M \qquad ( Equation 11 )$$

M is the number of body pressure sensors for each raising and lowering link module.

Here, M is the number of body pressure sensors for each raising and lowering link module, and Pij is a body pressure measured at a j-th sensor of an i-th raising and lowering link module.

In addition, in each raising and lowering link module, a pressure ratio ĥᵢ may be obtained by Equation 12 below.

$${R}_{i} = \frac{{\sum }_{j = 1}^{M} {P}_{ij}}{{\sum }_{i = 1}^{N} {\sum }_{j = 1}^{M} {P}_{ij}} , i = 1 , 2 , … , N , j = 1 , 2 , … , M \qquad ( Equation 12 )$$

Here, Pij is a body pressure measured at a j-th sensor of an i-th raising and lowering link module.

Through the above-described process, the fuzzy body pressure control of each raising and lowering link module may be performed by the fuzzy controller to prevent a pressure ulcer.

Meanwhile, in a case where the body pressure control performed by each body pressure sensor in the first step fails (for example, in a case when a patient is severely overweight), the duration control is performed as the second step.

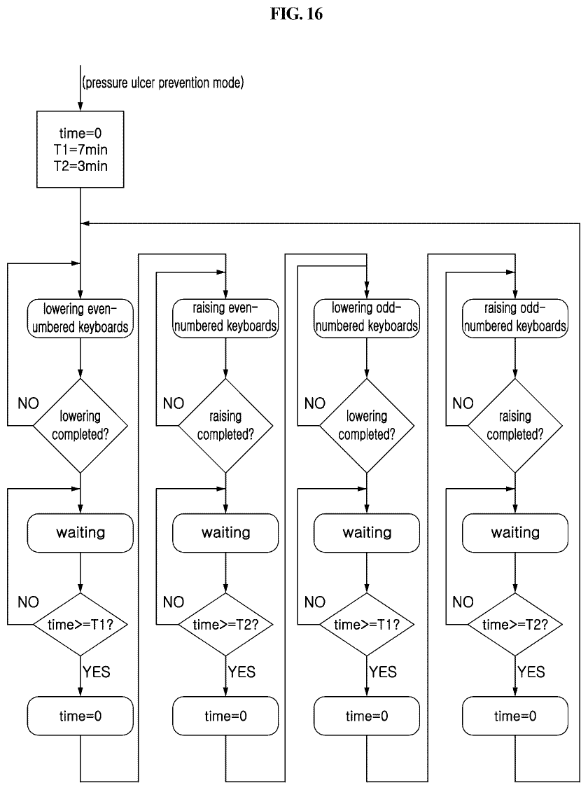

FIG. 16 is a flowchart illustrating the duration control according to the exemplary embodiment of the present disclosure.

Each of the raising and lowering link modules (i.e., each keyboard, 300) of the pressure ulcer prevention bed repeatedly performs pressure ulcer prevention movements for a preset time, and odd-numbered raising and lowering link modules and even-numbered raising and lowering link modules alternately perform the raising and lowering movements.

That is, after a preset certain period of time has elapsed, the odd-numbered or even-numbered raising and lowering link modules are lowered to set body pressure to zero, or when the raising and lowering link modules are raised again, it is controlled for the body pressure duration to be reduced much shorter than that of an ordinary body position change time (e.g., two hours), thereby in principle preventing a pressure ulcer to occur. In addition, the raising and lowering link modules raised are automatically lowered again after the critical duration has elapsed, so that the body pressure of a corresponding contact part pressed against a human body becomes zero.

In principle, it is known that a body position change time for controlling body pressure should be within two hours, but as much as possible, the shorter the body position change time, the better. It is defined such that during T1 (=7 minutes), the even-numbered or odd-numbered lowering link modules are lowered, and during T2 (=3 minutes), the lowered raising and lowering link modules are raised again to maintain the raising and lowering link modules forming a flat bed. A pressure ulcer control program was written so that the raising and lowering link modules repeat raising and lowering movements every T1+T2 (=10 minutes).

As a result of the conventional research on applied intensity and pressure duration with respect to the occurrence of pressure ulcers, it is known that histological change to a human body occurs when a pressure of 100 mmHg or more is applied for 30 minutes or a pressure of 45 to 80 mmHg is applied for one hour or more.

In this case, the two hours, which is the body position change time generally recommended in clinical practice because repetitively applied pressure causes mild histological changes at 45 to 60 mmHg and moderate or severe histological changes at 80 mmHg, is not sufficient to prevent a pressure ulcer, and thus, in order to effectively prevent the pressure ulcer, it is actually recommended to reduce pressure between a patient's body and a bed surface to less than 45 mmHg by shortening a period of body position change or using a special mattress.

In particular, it is worth noting that although the conventional automatic pressure control cylindrical air mattress reduces pressure enough to prevent a pressure ulcer, a mattress should be used together for a hip area. There is currently no clear standard limit for a lower limit value of pressure causing a pressure ulcer, but it is known that the risk of developing the pressure ulcer increases when the pressure greater than or equal to an approximate critical pressure of 60 mmHg is generated.

Accordingly, when it is based on the previous research results, a histological change occurs just in 30 minutes at 100 mmHg, so the embodiment of the present disclosure changes body pressure much earlier, i.e., every 10 minutes, than the 30 minutes, thereby preventing the histological change.

Accordingly, T1 and T2 is allowed to be set in a user interface unit 1100, so that a pressure ulcer prevention cycle is changeable, and thus it is required to optimally set the T1 and T2 for a patient's comfort and effective pressure ulcer prevention.

That is, the duration control in the second step is a control method of forcibly performing the raising and lowering movements and duration of the raising and lowering movements of each raising and lowering link module (i.e., each keyboard, 300) for a preset time.

The process of the above duration control may be summarized as follows.

As an example, the odd-numbered or even-numbered raising and lowering link modules (the keyboards, 300) are alternately forcibly raised and lowered for a preset time. When the odd-numbered or even-numbered raising and lowering link modules 300 are lowered, the body pressure applied to the lowered raising and lowering link modules 300 becomes zero.

Subsequently, after the predetermined period of time has elapsed, a process of raising the lowered raising and lowering link modules 300 upward again is repeated.

In this case, an alternating raising and lowering time (T1+T2) of the odd-numbered and even-numbered raising and lowering link modules may be set to proceed within 10 minutes.

Therefore, in a case where the duration control is performed as above, the occurrence of a pressure ulcer may be fundamentally prevented by distributing the body pressure of a human body.

That is, in the first step, body pressure sensors are attached to each raising and lowering link module, a body pressure is directly sensed by each body pressure sensor, and body pressure sensing information is transmitted to the motion control unit to appropriately control raising and lowering of the raising and lowering link modules through fuzzy body pressure control (i.e., artificial intelligence control), so that the body pressure of the human body may be distributed, whereby a pressure ulcer may be prevented.

In addition, as an exceptional case, when the fuzzy body pressure control in the first step fails due to overweight and the like, a plurality of raising and lowering link modules is raised and lowered by alternating between odd-numbered ones and even-numbered ones for a preset time within a body pressure critical duration for the movements of the plurality of raising and lowering link modules through the duration control in the second step, whereby it is possible to completely prevent a pressure ulcer.

Accordingly, in the present disclosure, unlike a simple alternating raising and lowering method in accordance with the time commonly used in conventional pressure ulcer mattresses or pneumatic cylinder-controlled medical beds, it is possible to confirm the effect of being able to independently distribute and control body pressure applied differently to each body part without compromising the comfort of a user with limited mobility.

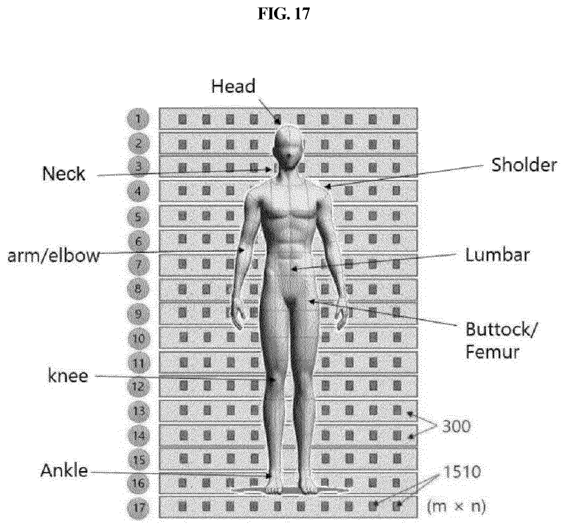

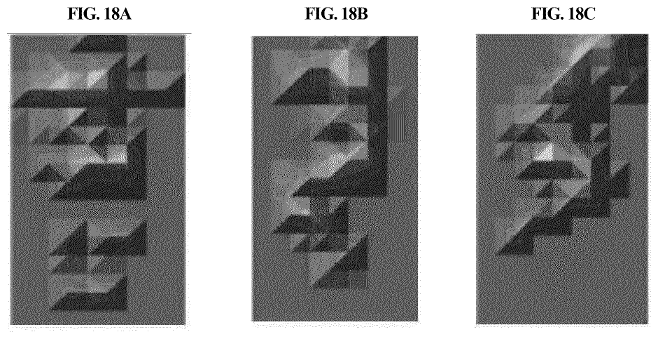

FIG. 17 is a plan view illustrating a use state of the pressure ulcer prevention bed using the body pressure sensors of the present disclosure. FIGS. 18A to 18C is an exemplary view of a body pressure distribution map according to the present disclosure.

Referring to FIGS. 17 and 18A˜18C, as described above, a pressure ulcer prevention bed using body pressure sensors according to the present disclosure includes: a plurality of raising and lowering link modules arranged in parallel adjacent to an upper surface of a bed support unit and whose raising and lowering is independently controlled by parallelogram links; the body pressure sensors provided in the respective raising and lowering link modules and constantly collecting body pressure sensing information for each part of a human body applied to each raising and lowering link module; a servo motor provided in the parallelogram links of each raising and lowering link module; and a motion control unit connected to the servo motor through wired and wireless communication and independently controlling each raising and lowering link module by using the body pressure sensing information individually transmitted from the body pressure sensors.

In this case, the body pressure sensors each consists of a plurality of unit body pressure sensors 1510-1, . . . , and 1510-n, and each unit body pressure sensor is arranged with adjacent unit body pressure sensors in a row at predetermined intervals along an upper surface of each raising and lowering link module.

That is, the body pressure sensors are distributed at predetermined intervals on the plurality of raising and lowering link modules for forming the upper surface of a bed support unit. Among these body pressure sensors, in a specific body pressure sensor pressed by a user's body weight, body pressure sensing information on body pressures applied through corresponding body parts is measured, quantified, and transmitted to the motion control unit. Accordingly, the user's body pressure maps according to various body postures may be generated as shown in FIG. 18 on the basis of the body pressure sensing information measured through each body pressure sensor.

For example, through CAN communication, a main controller (i.e., a motion control unit) transmits a command in angles on height values of the respective raising and lowering link modules (the keyboards) to the N motor servo drivers, and reads actual height values from the motor servo drivers. Likewise, M body pressure sensor values of each keyboard are read by the sensor controller and transmitted to the main controller through CAN communication. In this case, the larger the NM number of the sensor values, the higher the resolution of a body pressure map.

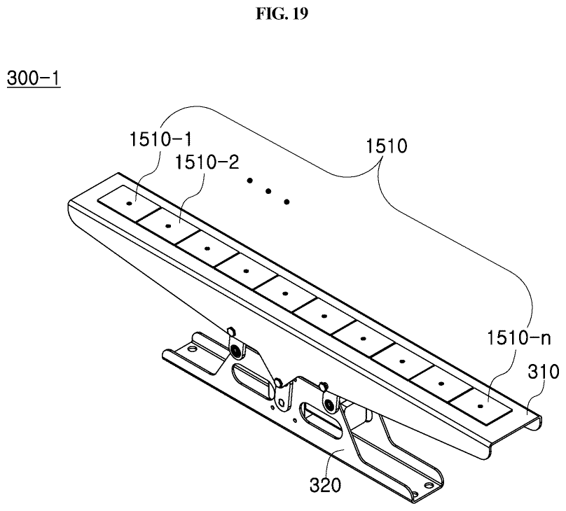

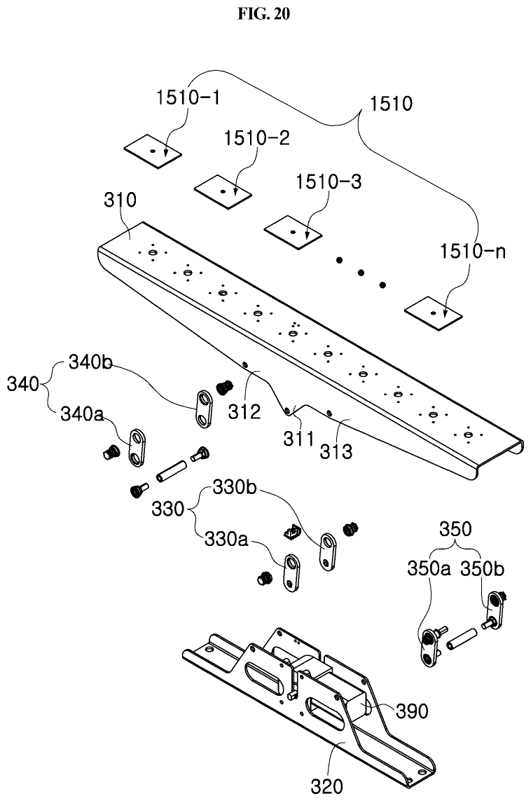

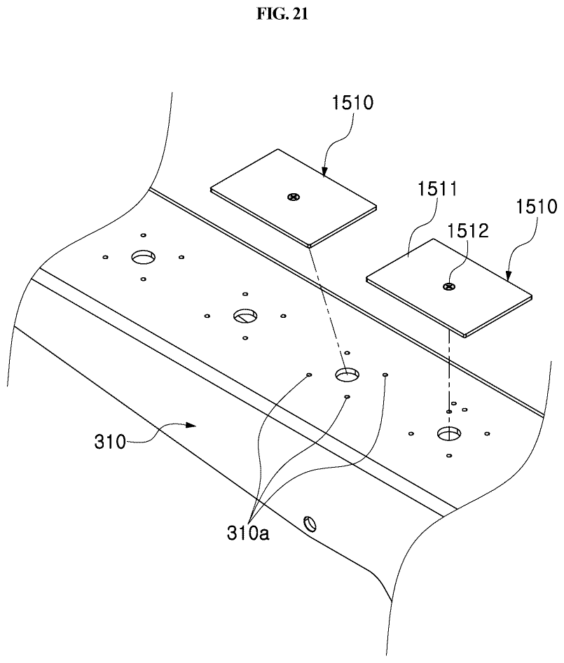

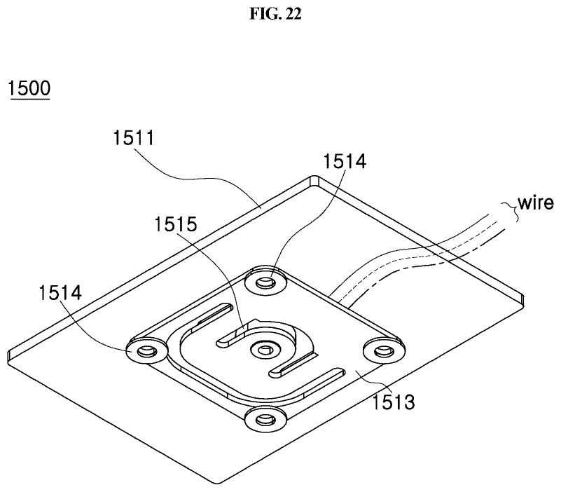

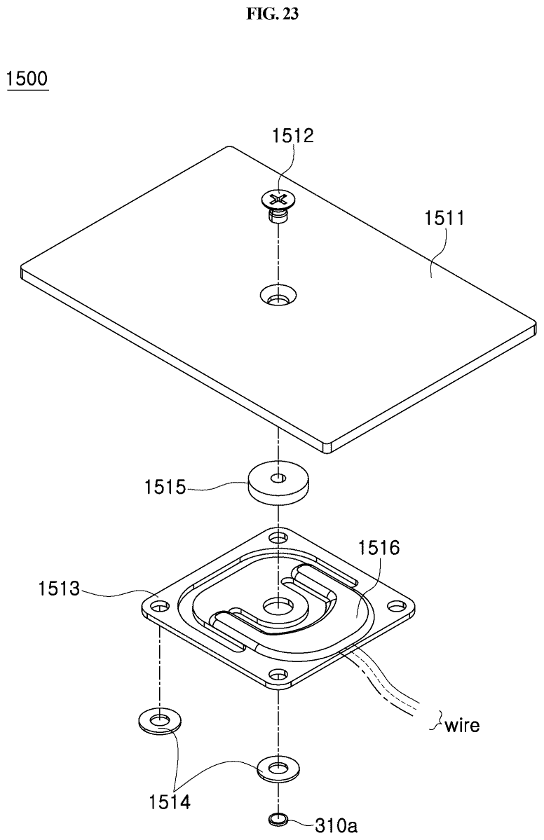

FIG. 19 is an enlarged perspective view illustrating a main part of a raising and lowering link module according to a yet another exemplary embodiment of the present disclosure. FIG. 20 is an exploded perspective view of FIG. 19. FIG. 21 is an enlarged view of a main part of FIG. 20. FIG. 22 is a bottom perspective view illustrating a body pressure sensor of FIG. 21. FIG. 23 is an exploded perspective view of FIG. 22.

In addition, with reference to FIGS. 19 to 23, a yet another exemplary embodiment in which body pressure sensor 1510 is coupled to each raising and lowering link module (each keyboard, 300-1) of the present disclosure is specifically illustrated.

First, the body pressure sensor 1510 with unit sensors at predetermined intervals is attached to the upper surface of each raising and lowering link module 300-1. For example, for each raising and lowering link module 300-1, ten unit body pressure sensors 1510-1, . . . , 1510-n are arranged adjacent to each other while being spaced at the predetermined intervals along a longitudinal direction of each raising and lowering link module 300-1.

Therefore, through the plurality of unit body pressure sensors 1510-1 . . . , 1510-n that are densely distributed on the upper surface of a bed, the body pressure sensing information transmitted may be received from the corresponding unit body pressure sensors (1510-1, . . . , 1510-n) contacting a user's body parts, thereby enabling checking of the intensity of body pressure for each body part, the duration of body pressure, the user's posture in real time, and the like.

In addition, each unit body pressure sensor 1510-1, . . . , 1510-n includes: a sensing plate 1511 arranged with adjacent sensing plates in the row at the predetermined intervals along the upper surface of each of the raising and lowering link modules 300-1; and a load cell 1513 formed in a plate shape and provided between the sensing plate 1511 and each raising and lowering link module 300-1.

According to the exemplary embodiment, in order to measure body pressure more accurately, the body pressure sensor 1510 using the load cells 1513, i.e., strain gauges, was manufactured. A strain gauge is a device used to measure the influence of external force or torque. The half-bridge type load cells using the strain gauges were manufactured in the form of a strip, and were mounted on each segment of a bed (see FIG. 17).

In addition, first spacers 1514 are further included between the load cell 1513 and the upper surface of each raising and lowering link module 300-1, and as a body pressure is added to an upper surface of the sensing plate 1511, the body pressure sensing information is measured within a thickness range of each first spacer 1514 while the load cell 1513 is elastically pressed. That is, in a case where the load cell 1513 is pressed by each first spacer 1514, only a range as much as the thickness of each first spacer 1514 is pressed to cause deformation and no further deformation beyond that, thereby preventing the load cell 1513 from being damaged due to excessive weight and excessive deformation.

For example, each first spacer 1514 may be a washer, and a first spacer 1514 may be screwed into a fastening hole 310a (see FIG. 21) formed on the upper surface of an upper link frame 310 of the raising and lowering link module 300-1.

In addition, according to the exemplary embodiment, between a sensing plate and a load cell, each unit body pressure sensor further includes: a second spacer 1515 provided between central parts of the sensing plate 1511 and the load cell 1513 and configured to maintain a gap between the sensing plate 1511 and the load cell 1513; a fastening screw 1512 for integrally coupling the central parts of the sensing plate 1511, the second spacer 1515, and the load cell 1513; and a silicone resin layer 1516 formed on an upper surface of the load cell to surround the second spacer and configured to provide elasticity, so as to allow the sensing plate 1511 to be elastically pressed or restored to an original position thereof.

In addition, in a process of attachment, first, the second spacer 1515 is caulked and fixed to the load cell 1513, four first spacers 1514 are placed on the upper surface of each upper link frame 310, the load cell 1513 is placed on top of each first spacer 1514, and the upper link frame 310 and the load cell 1513 are fixed by screw coupling. Thereafter, assembly is completed by fixing the sensing plate 1511 to the second spacer 1515 with the fastening screw 1512.

Meanwhile, the load cell 1513 is composed of a 3-wire half bridge, so (+) and (−) lines thereof are tied together in common and connected to each other, and only a middle signal line is connected to a sensing board (not shown), thereby completing a circuit configuration.

In addition, the present disclosure is not limited to just exemplary embodiments described above, and the same effect may be created even when the detailed configuration, number, and arrangement structure of the devices are changed, so it is clearly states that those skilled in the art will clearly state that various additions, deletions, and modifications may be made within the scope of the technical idea of the present disclosure.

1. A pressure ulcer prevention bed using body pressure sensors, the pressure ulcer prevention bed comprising:
a plurality of raising and lowering link modules arranged in parallel adjacent to an upper surface of a bed support unit and whose raising and lowering is independently controlled by parallelogram links;
the body pressure sensors provided in the respective raising and lowering link modules and constantly collecting body pressure sensing information for each part of a human body applied to each raising and lowering link module;
a servo motor provided in the parallelogram links of each raising and lowering link module; and
a motion control unit connected to the servo motor through wired and wireless communication and independently controlling each raising and lowering link module by using the body pressure sensing information individually transmitted from the body pressure sensors,
wherein the body pressure sensors each consists of a plurality of unit body pressure sensors, and each unit body pressure sensor is arranged with adjacent unit body pressure sensors in a row at predetermined intervals along an upper surface of each raising and lowering link module.

2. The pressure ulcer prevention bed of claim 1, wherein each unit body pressure sensor comprise:
a sensing plate arranged with adjacent sensing plates in the row at the predetermined intervals along the upper surface of each raising and lowering link module; and
a load cell formed in a plate shape and provided between the sensing plate and each raising and lowering link module.

3. The pressure ulcer prevention bed of claim 2, wherein each unit body pressure sensor further comprises:
first spacers between the load cell and the upper surface of each raising and lowering link module, and
as a body pressure is applied to an upper surface of the sensing plate, the body pressure sensing information is measured within a thickness range of each first spacer while the load cell is elastically pressed.

4. The pressure ulcer prevention bed of claim 2, wherein, between the sensing plate and the load cell, each unit body pressure sensor further comprises:
a second spacer provided between central parts of the sensing plate and the load cell, and configured to maintain a gap between the sensing plate and the load cell;
a fastening screw for integrally coupling the central parts of the sensing plate, the second spacer, and the load cell to each other; and
a silicone resin layer formed on an upper surface of the load cell to surround the second spacer and configured to provide elasticity, so as to allow the sensing plate to be elastically pressed or restored to an original position thereof.

5. The pressure ulcer prevention bed of claim 1, wherein further comprises:
a user interface unit;
a host interface unit;
a multi-servo communication unit for connecting the servo motor provided in each raising and lowering link module through the wired and wireless communication; and
a multi-sensor communication unit for connecting the body pressure sensors provided in the raising and lowering link modules through the wired and wireless communication.

6. The pressure ulcer prevention bed of claim 1, wherein each body pressure sensor further comprises:
a body pressure sensing information processing unit for processing the body pressure sensing information.

7. The pressure ulcer prevention bed of claim 5, wherein the host interface unit is connected to the multi-servo communication unit and the multi-sensor communication unit through Controller Area Network (CAN) communication.

8. A control method of controlling a pressure ulcer prevention bed using body pressure sensors, the pressure ulcer prevention bed comprising: a plurality of raising and lowering link modules arranged in parallel adjacent to an upper surface of a bed support unit and whose raising and lowering is independently controlled by parallelogram links; the body pressure sensors provided in the respective raising and lowering link modules and constantly collecting body pressure sensing information for each part of a human body applied to each raising and lowering link module; a servo motor provided in the parallelogram links of each raising and lowering link module; and a motion control unit connected to the servo motor through wired and wireless communication and independently controlling each raising and lowering link module by using the body pressure sensing information individually transmitted from the body pressure sensors, the body pressure sensors each consisting of a plurality of unit body pressure sensors, and each unit body pressure sensor being arranged with adjacent unit body pressure sensors in a row at predetermined intervals along an upper surface of each raising and lowering link module,
wherein, in a case where the body pressure sensing information collected from the body pressure sensors of the plurality of respective raising and lowering link modules is read by the motion control unit and body pressure sensing information transmitted from a body pressure sensor of a specific raising and lowering link module approaches 32 mmHg that is a pressure ulcer critical pressure at which a pressure ulcer is likely to occur, just before the specific raising and lowering link module with a pressure having come close to the pressure ulcer critical pressure reaches the pressure ulcer critical pressure, not only movements are stopped for a preset certain residence time after lowering only the specific raising and lowering link module with the pressure having come close to the pressure ulcer critical pressure from an initial first vertical position (h1) where all the raising and lowering link modules comprising the specific raising and lowering link module are all parallel to a second vertical position (h2) that is relatively lower than the first vertical position (h1), but also the specific raising and lowering link module stopped at the second vertical position (h1) is raised again to the initial first vertical position (h1) after the certain residence time has elapsed, so that control is independently performed for the raising and lowering movements and vertical positions of each raising and lowering link module according to the body pressure sensing information, and alternatively, in a case of a failure where the body pressure sensing information collected from the body pressure sensors of each raising and lowering link module is read to independently control the movements and positions of the specific raising and lowering link module, duration control as a next step is automatically performed to repeatedly control the raising and lowering movements of each raising and lowering link module for a preset time.

9. The control method of claim 8, wherein, in the motion control unit, the maximum body pressure is calculated in each raising and lowering link module by Equation 1 below:
$${P}_{mi} = max {p}_{ij} , j = 1 , 2 , … , M \qquad ( Equation 1 )$$
(where, Pmi is the maximum pressure of a raising and lowering link module i, Pij is a body pressure measured at a j-th sensor of the i-th raising and lowering link module, and M is the number of body pressure sensors in each raising and lowering link module),
an error (ei) is calculated by Equation 2 below:
$${e}_{i} = min ( {p}_{mi} - {p}_{cu} , 0 ) , i = 1 , 2 , … N \qquad ( Equation 2 )$$
(where, pₛᵥ is a target pressure value, pₘᵢ is the maximum pressure of the keyboard i, and uᵢ is a keyboard input),
a total error (eRMS) is calculated by Equation 3 below:
$${e}_{RMS} = \sqrt{\frac{{\sum }_{i = 1}^{N} {e}_{i}^{2}}{N}} , i = 1 , 2 , … , N \qquad ( Equation 3 )$$
(where, N is the number of raising and lowering link modules), and
the motion control unit independently controls the raising and lowering operation and vertical positions of each raising and lowering link module so that a total error value calculated by the above Equations converges to zero.

10. The control method of claim 8, wherein the motion control unit uses a Proportional-Integral (PI) control method or an Integral-Proportional (IP) control method.

11. The control method of claim 8, wherein the duration control divides the raising and lowering link modules into odd-numbered raising and lowering link modules and even-numbered raising and lowering link modules, and then repeatedly and alternately controls the raising and lowering operation of the odd-numbered raising and lowering link modules and even-numbered raising and lowering link modules for a preset lowering time (T1) and a preset raising time (T2).

12. The control method of claim 11, wherein a sum of the lowering time (T1) and the raising time (T2) is within 30 minutes.
